(19) United States
(12) Patent Application Publication
(10) Pub. No.: US 2008/0095801 A1
(43) Pub. Date: Apr. 24, 2008

(54) Homeopathic agent for the prophylaxis of diseases caused by parasites and method of producing and using the homeopathic agent

(76) Inventor: Andrea Weineck, Heidelberg/zgh (DE)

Correspondence Address: LERNER GREENBERG STEMER LLP, P O BOX 2480, HOLLYWOOD, FL, 33022-2480, US

(21) Appl. No.: 11/820,150

(22) Filed: Jun. 18, 2007

Related U.S. Application Data
(63) Continuation of application No. PCT/EP05/13442, filed on Dec. 14, 2005.

(30) Foreign Application Priority Data
Dec. 18, 2004 (DE) 10 2004 061 011.8

(51) Int. Cl. A61K 39/02; A61P 31/04

(52) U.S. Cl. 424/236.1

(57) ABSTRACT

A homeopathic agent containing Ledum palustre in the form of a homeopathic dilution in an inert carrier material and at least one secretion from a parasite, a sufferer of a disease caused by parasites, or from an intermediate host in the form of a homeopathic dilution in an inert carrier material.

### CROSS-REFERENCE TO RELATED APPLICATION

This is a continuing application, under 35 U.S.C. § 120, of copending international application No. PCT/EP2005/013442, filed Dec. 14, 2005, which designated the United States; this application also claims the priority, under 35 U.S.C. § 119, of German patent application DE 10 2004 061 011.8, filed Dec. 18, 2004; the prior applications are herewith incorporated by reference in their entirety.

### BACKGROUND OF THE INVENTION

### Field of the Invention

The present invention relates to a homeopathic agent containing Ledum palustre in a form of a homeopathic dilution in an inert carrier material and at least one secretion from a parasite, a sufferer of a disease caused by parasites, or else from an intermediate host, in a form of a homeopathic dilution in the inert carrier material.

The present invention further relates to a method for preparing the homeopathic agent of the invention and its use to prevent infectious diseases caused by parasites, and generally its use for the prevention of parasite and insect bites or animal bites. Moreover, the present invention also encompasses items of clothing, jewelry, animal collars, crystals or decorative elements containing the homeopathic agent of the invention.

Parasites such as fleas, mosquitoes, ticks, spiders, worms, bees, wasps or hornets, for example, can cause dangerous infectious diseases in humans and animals. These diseases can be induced either by such parasites passing their own venom on to other creatures by a bite, or else it is also possible that their bite releases viruses, bacteria or other causative agents into other creatures where they are able then to cause dangerous infectious diseases. In this case, such parasites are only intermediate hosts for the causative agents of, inter alia, malaria, Lyme disease or meningoencephalitis.

Agents and devices for prevention of coming into contact with such parasites and thus for the prophylaxis of infectious diseases have long been known. U.S. Pat. Nos. 5,038,718 and 5,155,950 each describe belts as a barrier against ticks, which are intended to protect humans wearing them from being bitten by these animals. Belts of this kind contain, inter alia, adhesive surfaces, chemically treated surfaces or electrically conducting surfaces and are of relatively complex design. U.S. Pat. No. 5,381,557 further discloses a band having an adhesive surface with which, inter alia, ticks, spiders or other insects can be repelled. The belt, which is of relatively complex design, likewise has adhesive surfaces.

U.S. Pat. No. 6,141,802 relates to tubular structures for repelling ticks, which ought to be worn by the user inside trousers or socks, and have been treated for this purpose with tick-repellent chemicals.

The above-mentioned options for repelling in particular ticks and spiders are all based on items of clothing of relatively complex design and contain as a rule chemicals to repel such parasites as well as chemical adhesives. However, many users wish for a very effective and at the same time mild and gentle agent against such parasites that are simple to use.

### SUMMARY OF THE INVENTION

It is accordingly an object of the invention to provide a homeopathic agent for the prophylaxis of diseases caused by parasites and method of producing and using the homeopathic agent which overcome the above-mentioned disadvantages of the prior art methods and devices of this general type, which provides an agent for repelling the parasites mentioned and/or insects or animals in general, which is extremely easy to handle and fulfills the object in a mild and gentle fashion and without side effects.

Accordingly, a homeopathic agent or an active substance combination has been developed, contains a) Ledum palustre in the form of a homeopathic dilution in an inert carrier material and b) at least one secretion from a parasite, a sufferer of a disease caused by parasites, or else from an intermediate host in the form of a homeopathic dilution in an inert carrier material.

Ledum palustre, also referred to as marsh tea or wild rosemary, which is used in the homeopathic agent of the invention is an agent utilized in homeopathy and known, inter alia, for its antiseptic properties. It is preferably used in a homeopathic dilution according to the decimal scale from D1 to D1 000 000, preferably from D10 to D10 000, more preferably from D15 to D5000 and most preferably from D20 to D2000. According to the centesimal scale, the homeopathic dilution is preferably C1 to C1 000 000, ideally C10 to C10 000, more preferably C15 to C5000 and most preferably C20 to C2000. Ledum palustre is available in various dilutions from homeopathic retailers.

Homeopathic dilutions are generally prepared according to the decimal or centesimal scale. To this end, the basic materials such as plants, parts of or secretions from animals, metals or minerals are first comminuted and then dissolved in water and/or alcohol. Some insoluble substances, metals for example, are often pulverized, then mixed with powdered lactic sugar and thus brought into an alcohol-soluble form. The basic material obtained in this way is then dissolved in a water/alcohol mixture at a specific ratio. This mixing ratio depends in most cases on the solubility behavior of the respective substances. The mixture obtained in this way is then allowed to swell for a certain time, several hours up to several months for example. What is called the “mother tincture” is obtained from this mixture by, inter alia, extraction, screening, filtration, pouring off, decanting or pressing. It is then again dissolved in a mixture of alcohol and water until the desired potency has been attained. Homeopathic dilutions are prepared as follows through what is called potentization.

A D1 dilution (decimal 1) is obtained by adding 9 drops of a solution of usually distilled water and alcohol (ethanol) to 1 drop of a particular mother tincture. The alcohol content of this solution again depends decisively on the solubility behavior of the substances used and is often in the range from 5 to 95% by weight, especially in the range from 10 to 90% by weight. The solution obtained in this way is then succussed (shaken) several times. This will give what is referred to as a D1 dilution. If 1 drop of the D1 dilution is combined with 9 drops of an alcoholic aqueous solution (in the same mixing ratio as for D1 dilutions) and subsequently succussed (shaken) several times, this will result in what is referred to as a D2 dilution. A D3 dilution is obtained by removing one drop from a D2 dilution and dissolving it in 9 drops of an alcoholic aqueous solution, followed by repeated succussion. In the case of a D20 dilution, this process is carried out, starting from the mother tincture, a total of 20 times.

For what is referred to as C1 dilution (centesimal 1), one drop is removed from the mother tincture and added to 99 drops of a water/alcohol mixture which is then succussed several times. One drop of this C1 dilution and 99 drops of an alcoholic aqueous solution then give what is referred to as C2 dilution after appropriate succussion. This process is always to be repeated until the desired dilution has been attained, that is in the case of a C200 dilution, for example, a total of 200 times.

The homeopathic dilution obtained in this way is used either directly in the form of drops or else is admixed with a solid inert carrier material, with lactic sugar, starch or other types of sugar, for example, and proffered as homeopathic medicine in the form of globules (pellets) or else in the form of tablets. In the case of homeopathic drops, the inert carrier material is usually solutions of alcohol and water.

Furthermore, the homeopathic agent of the invention contains at least one secretion from a parasite and/or a sufferer of a disease caused by parasites in the form of a homeopathic dilution in an inert carrier material, water and/or alcohol or lactic sugar, starch or other types of sugar, for example. The secretion from a sufferer (a human or an animal) of a disease caused by parasites includes various fluids excreted by the body or solid body parts of a creature infected through a bite (fleas, spiders or ticks), a sting (bees, wasps, bumblebees or hornets) or some other contact (worms). The secretion is dissolved in a mixture of water and alcohol and appropriately potentized by continued dissolution and succussion. Constituent (b) preferably contains, inter alia, one secretion from a sufferer of Lyme disease and/or meningoencephalitis (from causative agents occurring both in Europe and in North America or other regions of the Earth) and/or the secretion from a sufferer of a mosquito bite or another disease transmitted by bites or stings.

Further preferred is a constituent (b) of the kind that contains a secretion from the Anopheles mosquito which transmits malaria, or a secretion from a sufferer of malaria. Malaria is a collective term for febrile infections caused by protozoa (monocellular organisms) that are transmitted through the bite of the Anopheles mosquito. The homeopathic agent of the invention may in this context contain one or several different secretions of the three commonly occurring forms of malaria, i.e. malaria tertiana (three-day fever), malaria quartana and malaria tropica.

It is preferable for secretion b) to be used in a homeopathic dilution according to the decimal scale from D1 to D1 000 000, ideally from D10 to D10 000, more preferably from D15 to D5000 and most preferably from D20 to D2000. According to the centesimal scale, the homeopathic dilution is preferably C1 to C1 000 000, ideally C10 to C10 000, more preferably C15 to C5000 and most preferably C20 to C2000. Secretion (b) is used either directly in the form of drops with water and/or alcohol as inert carrier materials or else applied to solid inert carrier materials, such as lactic sugar, starch or other types of sugar, and proffered in the form of globules or tablets.

It is available in this form from homeopathic retailers.

The homeopathic agent of the invention may further contains Delphinium staphysagria c) in the form of a homeopathic dilution in an inert carrier material. Delphinium staphysagria (also referred to as larkspur) is preferably used in a homeopathic dilution according to the decimal scale from D1 to D1 000 000, ideally from D10 to D10 000, more preferably from D15 to D5000 and most preferably from D20 to D2000. According to the centesimal scale, the homeopathic dilution for Delphinium staphysagria c) is C1 to C1 000 000, ideally C10 to C10 000, more preferably C15 to C5000 and most preferably C20 to C2000. Suitable carrier materials are likewise water and/or alcohol for example (if the pharmaceutical form is drops) or else lactic sugar, starch and other types of sugar (if c) is used in the form of globules or tablets). Delphinium staphysagria c) is available in the desired homeopathic dilutions from specialty retailers.

According to the inventive method for preparing the homeopathic agents of the invention, the homeopathic agents a, b) and, if appropriate, c) obtained by dilution and succussion are first applied to an inert carrier material (water and/or alcohol or inert solid carrier materials such as lactic sugar, starch or other types of sugar) and then stored as a mixture in the form of different globules, tablets, or as a solution in a suitable container or on a suitable material. The homeopathic agents of the invention are usually applied to the solid inert carrier materials by dripping the solution having the desired potency onto the carrier materials.

The individual substances of the homeopathic agent of the invention, i.e. a) and b), and if appropriate c), are preferably present in such quantitative ratios that Ledum palustre a) accounts for a proportion of from 10 to 90 percent by weight, especially from 15 to 80 percent by weight and most preferably from 20 to 70 percent by weight. Secretion b) accordingly is present in a quantitative ratio of from 10 to 90 percent by weight, especially from 15 to 80 percent by weight and more preferably from 20 to 70 percent by weight, with a) and b) each adding up to 100 percent by weight.

If Delphinium staphysagria c) is present in the homeopathic agent in addition to a) and b), the proportions of a), b) and c) are each 10 to 80 percent by weight, especially 15 to 70 percent by weight and most preferably 20 to 60 percent by weight, with the proportions of a), b) and c) each adding up to 100 percent by weight. It is preferred for each of the substances a), b) and, if appropriate, c) to be present in approximately equal proportions.

The homeopathic agent of the invention is stored in particular in the form of a solution of active substances a), b) and, if appropriate, c) or in the form of different globules or tablets which each contain an active substance a) and b) and, if appropriate, c, in a suitable container or on a suitable material.

It may be advisable to deposit the homeopathic agent in an item of clothing, a belt for example, a pouch, a band or another storage container to be worn on the body. This may of course also be an item of jewelry, for example a ring, a necklace, a necklet or else a brooch, in which the homeopathic agent is stored or onto the surface of which it is applied and sealed.

It is furthermore possible to apply the homeopathic agent of the invention in general directly to a carrier material or substrate, in particular to a crystal or decorative element such as a gemstone for example, i.e. not to store the inventive agent in a container. The energetic structure of the homeopathic agent of the invention is thus transferred onto the crystal or the decorative element.

The homeopathic agent of the invention is particularly suitable for preventing bites and stings of parasites, insects and other animals, and infectious diseases caused by parasites, such as for preventing, for example, Lyme disease, meningoencephalitis, that is to say tick-borne diseases, and diseases attributable to mosquito bites and fly bites, such as malaria or sleeping sickness (African trypanosomiasis), for example.

The appropriate effect unfolds through simply wearing the homeopathic agent on the body, without the need for it to be taken orally. It is therefore suitable primarily as a prophylactic agent for the prevention of bites and stings of parasites, of ticks or mosquitoes in particular, and thus for the prevention of transmission of relevant infectious diseases.

Other features which are considered as characteristic for the invention are set forth in the appended claims.

Although the invention is described herein as embodied in a homeopathic agent for the prophylaxis of diseases caused by parasites and method of producing and using the homeopathic agent, it is nevertheless not intended to be limited to the details described, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments.

### DETAILED DESCRIPTION OF THE INVENTION

The mode of action of the homeopathic agent according to the invention is to be illustrated in detail by the examples below.

### EXAMPLES

A total of 5 Ledum palustre a) globules in the homeopathic C200 dilution and applied to lactic sugar were combined with 5 Lyme disease b) globules (from the secretion from a sufferer of Lyme disease) in the C200 dilution and applied to lactic sugar, and 5 TBE b) globules (from the secretion from a sufferer of tick-borne encephalitis) in the C200 dilution and applied to lactic sugar and, furthermore, 5 Staphysagria c) globules in the C200 dilution and applied to lactic sugar in a pouch. With each of the globules weighing about 50 mg, the total weight of the in total 20 globules was about 1000 mg.

The globules used here had been obtained by appropriate potentization to C200 and succussion with the aid of a mixture of alcohol and water in accordance with the German Pharmacopeia.

In analogy to this, the same substances a) (dilution: C200), b) (Lyme disease, dilution: C200) and b) (TBE, tick-borne encephalitis, dilution: C200) and c) (dilution: C200), in the form of 4 tablets each weighing 260 mg, were combined in a small container.

The now described experiments were carried out with these homeopathic agents.

### Example 1

In a long-term experiment, the homeopathic agent of the invention was carried daily on the body over several years for the prophylaxis against ticks. The number of tick bites was reduced by about 70 to 98% by simply carrying the agent.

### Example 2

Furthermore, a huntsman who was exposed to about 20-30 tick bites each year, was given the homeopathic agent as a pouch to carry in his trouser pocket each day. Not a single tick bite has been found on him since then.

### Example 3

A farmer who had seriously fallen ill with Lyme disease as a result of a tick bite and suffered from severe joint pain was given the homeopathic agent of the invention for carrying it on him externally. He has not been bitten by ticks since he started having it on him at all times, except for a cycle tour when he did not carry the abovementioned active substance combination.

### Example 4

A German shepherd dog getting numerous tick bites since he was a puppy had the homeopathic agent of the invention attached to its collar. It has been virtually free from ticks since that time.

### Example 5

Another patient who enjoys gardening has been using the homeopathic agent of the invention for some time and has not had any tick bites since then, except when he forgot to carry the inventive agent in his pocket.

Examples 2-4 clearly show that the effectiveness of the homeopathic agent of the invention does not derive from a placebo effect, i.e. it is not brought on by the patient simply imagining it.

1. A homeopathic agent, comprising:
Ledum palustre in a form of a homeopathic dilution in an inert carrier material; and
at least one secretion from one of a parasite, a sufferer of a disease caused by parasites, and from an intermediate host, in a form of a homeopathic dilution in an inert carrier material.

2. The homeopathic agent according to claim 1, further comprising Delphinium staphysagria in a form of a homeopathic dilution in an inert carrier material.

3. The homeopathic agent according to claim 1, wherein said at least one secretion is from a sufferer of Lyme disease in the form of the homeopathic dilution in the inert carrier material.

4. The homeopathic agent according to claim 1, wherein said at least one secretion is from a sufferer of meningoencephalitis in the form of the homeopathic dilution in the inert carrier material.

5. The homeopathic agent according to claim 1, wherein said Ledum palustre in the homeopathic dilution is provided according to one of a decimal scale from D1 to D1 000 000 and a centesimal scale from C1 to C1 000 000.

6. The homeopathic agent according to claim 5, wherein said Ledum palustre in the homeopathic dilution is provide according to one of the decimal scale from D10 to D10 000 and the centesimal scale from C10 to C10 000.

7. The homeopathic agent according to claim 1, wherein said at least one secretion from one of the parasite and the sufferer of the disease caused by parasites in the homeopathic dilution is provided according to one of a decimal scale from D1 to D1 000 000 and to a centesimal scale from C1 to C1 000 000.

8. The homeopathic agent according to claim 7, wherein said at least one secretion from one of the parasite and the sufferer of the disease caused by parasites in the homeopathic dilution is provided according to one of the decimal scale from D10 to D10 000 and to the centesimal scale from C10 to C10 000.

9. The homeopathic agent according to claim 2, wherein said Delphinium staphysagria in the homeopathic dilution is provided according to one of a decimal scale from D1 to D1000 000 and a centesimal scale from C1 to C1000 000.

10. The homeopathic agent according to claim 9, wherein said Delphinium staphysagria in the homeopathic dilution is provided according to one of the decimal scale from D10 to D10 000 and the centesimal scale from C10 to C10 000.

11. The homeopathic agent according to claim 1, wherein
said inert carrier material is lactic sugar for globules or tablets; and
said inert carrier material is, selected from the group consisting of water and mixtures of water and alcohol, for drops.

12. A method for preparing a homeopathic agent, which comprises the steps of:
providing an inert carrier material;
providing Ledum palustre and at least one secretion from one of a parasite, a sufferer of a disease caused by parasites, and from an intermediate host, obtained by one of dilution and succession, and applied to the inert carrier material resulting in a mixture; and
storing the mixture in a form of one of different globules, tablets, a solution in a suitable container, and a solution on a suitable material.

13. The method for preparing the homeopathic agent according to claim 12, which further comprises adding in Delphinium staphysagria to the inert carrier material.

14. A substrate, comprising:
a substrate body; and
a homeopathic agent disposed on said substrate body, said homeopathic agent including:
Ledum palustre in a form of a homeopathic dilution in an inert carrier material; and
at least one secretion from one of a parasite, a sufferer of a disease caused by parasites, and from an intermediate host in a form of a homeopathic dilution in said inert carrier material.

15. The substrate according to claim 14, wherein said substrate body is selected from the group consisting of an item of clothing, jewelry and an animal collar.

16. A substrate, comprising:
a substrate body; and
an energetic structure disposed on said substrate body and corresponding to a homeopathic agent, said homeopathic agent including:
Ledum palustre in a form of a homeopathic dilution in an inert carrier material; and
at least one secretion from one of a parasite, a sufferer of a disease caused by parasites, and from an intermediate host in a form of a homeopathic dilution in said inert carrier material.

17. A carrier, comprising:
a carrier body selected from the group consisting of crystals and decorative elements;
a homeopathic agent disposed on said carrier body, said homeopathic agent including:
Ledum palustre in a form of a homeopathic dilution in an inert carrier material; and
at least one secretion from one of a parasite, a sufferer of a disease caused by parasites, and from an intermediate host in a form of a homeopathic dilution in said inert carrier material.

18. A method of using a homeopathic agent, which comprises the steps of:
providing a substrate having a homeopathic agent disposed on or in the substrate, the homeopathic agent including Ledum palustre in a form of a homeopathic dilution in an inert carrier material, and at least one secretion from one of a parasite, a sufferer of a disease caused by parasites, and from an intermediate host in a form of a homeopathic dilution in the inert carrier material;
placing the substrate on a user for preventing at least one of bites, stings and a transmission of infectious diseases caused by parasites.

19. The method according to claim 18, which further comprises placing the substrate on the user for preventing infectious diseases caused by a bite of ticks or mosquitoes.
